(19) United States
(12) Patent Application Publication
(10) Pub. No.: US 2007/0016170 A1
(43) Pub. Date: Jan. 18, 2007

(54) Infusion device with bolus alarm deactivation and method of using the same

(75) Inventor: Paul H. Kovelman, Simi Valley, CA (US)

Correspondence Address: MEDTRONIC MINIMED INC., 18000 DEVONSHIRE STREET, NORTHRIDGE, CA, 91325-1219, US

(73) Assignee: MEDTRONIC MINIMED, INC.

(21) Appl. No.: 11/171,010

(22) Filed: Jun. 29, 2005

(51) Int. Cl. A61K 9/22

(52) U.S. Cl. 604/890.1; 128/905

(57) ABSTRACT

A medical device for use with a patient includes at least one programmable medical device feature, at least one alarm to remind the patient about the at least one programmable medical device feature, and at least one reminder check to allow the patient to deactivate the at least one alarm while the at least one programmable medical device feature is being programmed. The at least one reminder check determines if the at least one alarm is scheduled to occur during a check ahead time that extends from the time the at least one programmable medical device feature is being programmed. The at least one programmable medical device feature may be at least one fluid delivery and the at least one fluid delivery may be at least one bolus delivery.

### FIELD OF THE INVENTION

This invention relates to infusion devices for delivering a fluid to a user and, in particular embodiments, to an infusion device that can deactivate a bolus alarm at the time a bolus is being delivered.

### BACKGROUND OF THE INVENTION

Traditionally, users have had to keep track of times to take boluses before or after meals to make sure they maintain their glucose levels at desired levels. Failure to take an insulin bolus either just prior or after a meal can result in abnormally high glucose levels, and can ultimately lead to long term complications or hospitalization.

To overcome this problem, users have used alarm clocks, logs or other reminder devices, to inform them on when to check to see if they took an anticipated bolus by a required time. Carrying multiple objects is cumbersome and inconvenient. Accordingly, it was proposed in U.S. Pat. No. 6,554,798 (which is incorporated by reference herein in their entirety) that a user settable alarm be incorporated into an infusion device, and this alarm could be set to different times to remind the user at predetermined times to take various actions.

In another variation, infusion devices with a user settable alarm for missed meal boluses, would check at the time when an alarm was to be provided to determine if a meal bolus had already been taken within a window ending at the alarm time. If a bolus had been taken within the time window, regardless of the type of bolus taken (i.e., whether or not it was taken for the purpose for which it was set), the alarm would be deactivated and no alarm would be provided at the specified time. Examples of this can be found in U.S. Pat. Nos. 6,650,951 and 6,744,350, both of which are incorporated by reference herein in their entirety. However, automatic deactivation of alarms can be problematic, since a user may not receive an alarm they still need to receive. Failure to act because of a missed or expected alarm could have serious health consequences if not attended to promptly.

### SUMMARY OF THE DISCLOSURE

It is an object of an embodiment of the present invention to provide an infusion device with improved missed bolus alarm deactivation, which obviates for practical purposes, the above mentioned limitations.

According to an embodiment of the invention, a medical device is for use with a patient. The medical device includes at least one programmable medical device feature, at least one alarm to remind the patient about the at least one programmable medical device feature, and at least one reminder check to allow the patient to deactivate the at least one alarm while the at least one programmable medical device feature is being programmed. In particular embodiments, the at least one reminder check determines if the at least one alarm is scheduled to occur during a check ahead time that extends from the time the at least one programmable medical device feature is being programmed. In additional embodiments, the at least one programmable medical device feature may be at least one fluid delivery. In other embodiments, the at least one fluid delivery may be at least one bolus delivery. In further embodiments, the at least one bolus delivery may include delivery of at least one of a normal bolus, square wave bolus, dual wave bolus, custom bolus, audio bolus, and/or profiled bolus. In still further embodiments, the at least one alarm may be at least one missed bolus alarm. In additional embodiments, the medical device may be a glucose monitor. In other embodiments, the medical device may be a glucose meter. In still additional embodiments, the medical device may be a PDA used as a programmer for the medical device.

In other embodiments, the medical device is an infusion device for infusion of fluid to a body of a patient. In particular embodiments, the programmable medical device feature may be fluid delivery, and, in additional embodiments, the fluid may be insulin. In further embodiments, the at least one fluid delivery may be at least one bolus delivery that includes delivery of at least one of a normal bolus, square wave bolus, dual wave bolus, custom bolus, audio bolus, and/or profiled bolus. In particular embodiments, the at least one alarm may be at least one missed bolus alarm.

In still other embodiments, the medical device is an infusion device that includes at least one programmable medical device feature, at least one alarm to remind the patient about the at least one programmable medical device feature, at least one reminder check to allow the patient to deactivate the at least one alarm while the at least one programmable medical device feature is being programmed, a drive mechanism, a processor to control the infusion device, a memory operatively coupled to the processor, at least one power supply, an input device operatively coupled to the processor to allow the patient to command the processor, a display device operatively coupled to the processor to provide visual information to the patient, and a housing. In additional embodiments, the programmable medical device feature may be at least one fluid delivery and, in some embodiments, the at least one fluid delivery may be at least one bolus delivery. In further embodiments, the at least one bolus delivery may include delivery of at least one of a normal bolus, square wave bolus, dual wave bolus, custom bolus, audio bolus, and/or profiled bolus. In still further embodiments, the at least one alarm may be at least one missed bolus alarm.

In other embodiments, a method of deactivating an alarm in a medical device includes the steps of programming at least one programmable medical device feature, accessing at least one reminder check to determine if at least one alarm is scheduled to occur during a check ahead time that extends from the time the at least one programmable medical device feature is being programmed, and deactivating the at least one alarm prior to completing programming of the at least one programmable medical device feature. In particular embodiments, the at least one programmable medical device feature may be at least one fluid delivery. The at least one fluid delivery may be least one bolus delivery. In further embodiments, the at least one bolus delivery may include delivery of at least one of a normal bolus, square wave bolus, dual wave bolus, custom bolus, audio bolus, and/or profiled bolus. In additional embodiments, the at least one alarm may be at least one missed bolus alarm. In still further embodiments, the method may be for deactivating a missed bolus alarm in an infusion device.

In additional embodiments, a medical device is for use with a patient. In these embodiments, the medical device includes programming means for controlling at least one programmable medical device feature in the medical device, alarm means for reminding the patient about the at least one programmable medical device feature, and reminder means for allowing the patient to deactivate the alarm means while the programming means is being used to program the at least one programmable medical device feature. In particular embodiments, the reminder means determines if the alarm means is scheduled to occur during a check ahead time that extends from the time the programming means is programming the at least one medical device feature. In other embodiments, the medical device is an infusion device. In particular embodiments, the programming means may include bolus means for programming at least one bolus delivery as the at least one programmable medical device feature. In still other embodiments, the alarm means may include missed bolus alarm means for notifying the patient of a missed bolus delivery. In still further embodiments, the reminder means is for allowing the patient to deactivate the missed bolus alarm means while the at least one bolus delivery is being programmed. In additional embodiments, the reminder means determines if the missed bolus alarm means is scheduled to occur during a check ahead time that extends from the time the at least one bolus delivery is being programmed.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

A detailed description of embodiments of the invention will be made with reference to the accompanying drawings, where like numerals designate corresponding parts in the several figures.

FIG. 1 is a perspective view of an embodiment of an infusion device in accordance with an embodiment of the present invention.

FIG. 2 is a simplified schematic view of the embodiment of FIG. 1.

FIG. 3 is a flow diagram of an aspect of an embodiment of the present invention.

FIG. 4 is a flow diagram of another aspect of an embodiment of the present invention.

FIG. 5 is a flow diagram of a further aspect of an embodiment of the present invention.

### DETAILED DESCRIPTION OF THE PREFERRED EMBODIMENTS

As shown in the drawings for purposes of illustration, the invention is embodied in an infusion device with a bolus alarm feature. In particular embodiments of the present invention, the bolus alarm is deactivated at the time a bolus is administered. In further embodiments, the infusion device is an external infusion device. However, it will be recognized that further embodiments of the invention may be used in monitoring devices, test strip meters, PDAs, computers, implantable pumps, or the like.

As shown in the drawings for purposes of illustration, the invention is embodied in an external infusion device for infusion of a liquid, such as medication, chemicals, enzymes, antigens, hormones, vitamins or the like, into a body of a user. In particular embodiments of the present invention, the external infusion device is an external infusion pump, which includes an optional RF programming capability, a bolus capability and/or alarm capability. Embodiments are directed towards use in humans; however, in alternative embodiments, the external infusion devices may be used in animals.

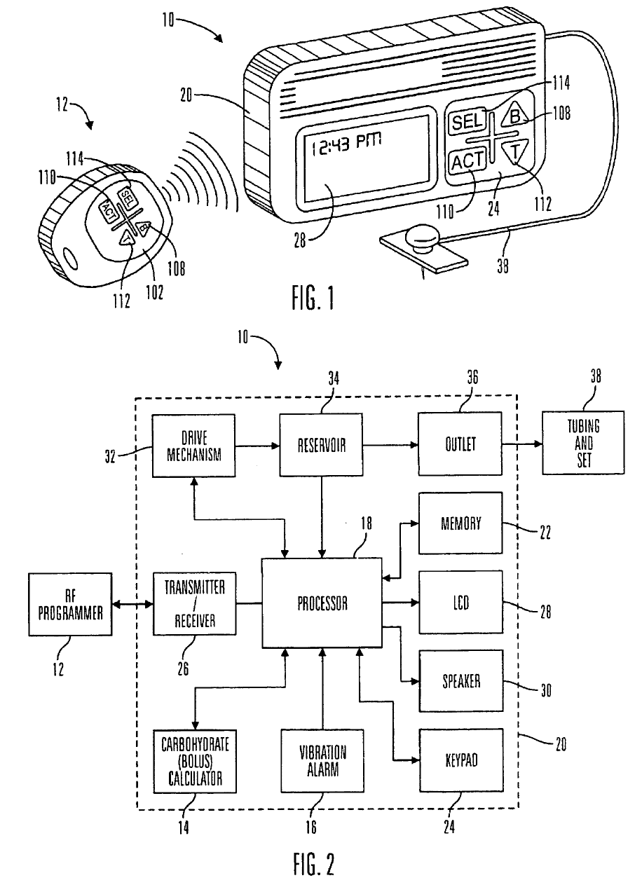

As illustrated in FIGS. 1 and 2, embodiments of the external infusion device 10 include an optional remote RF programmer 12, a bolus capability 14 and/or an alarm 16. The RF programmer 12 and bolus capability 14 communicate with a processor 18 contained in a housing 20 of the external infusion device 10. The processor 18 is used to run programs and control the external infusion device 10, and is connected to an internal memory device 22 that stores programs, historical data, user defined information and parameters. In particular embodiments, the memory device is a Flash memory and SRAM; however, in alternative embodiments, the memory device 22 may include other memory storage devices such as ROM, DRAM, RAM, EPROM, dynamic storage such as other flash memory, energy efficient hard-drive, or the like. In other embodiments, the external infusion device 10 is an external infusion pump that is programmed through a keypad 24 on the housing 20 or by commands received from the RF programmer 12 through a transmitter/receiver 26. Feedback from the external infusion device 10 on status or programming changes are displayed on an LCD 28 and/or audibly through a speaker 30. In alternative embodiments, the keypad 24 may be omitted and the LCD 28 may be used as a touch screen input device or the keypad 24 may utilize more keys or different key arrangements then those illustrated in the figures. The processor 18 is also coupled to a drive mechanism 32 that is connected to a fluid reservoir 34 containing fluid that is expelled through an outlet 36 in the reservoir 34 and housing 20, and then into a body of a user through tubing and a set 38. In further alternative embodiments, the keypad 24, LCD 20, and speaker 24 may be omitted from the external infusion device, and all programming and data transfer is handled through the RF programmer 12.

Generally, in particular embodiments of the external infusion device 10 are an external insulin pump having the capability to deliver 0 to 35 Units/hour in basal rates and up to 25.0 Units per meal bolus of U-100 Insulin. In alternative embodiments, the external pump delivers other concentrations of insulin, or other liquids, and may use other limits on the delivery rate.

To deliver a bolus with the keypad the user uses the keypad 24 and keys 108, 110, 112 and/or 114 to program and/or deliver one or more bolus types through a single touch key or by the use of one or more menus. In alternative embodiments, the user can program and/or deliver a bolus with the optional RF programmer 12.

Examples of how to program and/or deliver a bolus and the different types of boluses can be found in U.S. Pat. No. 6,554,798 issued on Apr. 29, 2003 to Mann et al., and entitled “External Infusion Device with Remote Programming, Bolus Estimator and/or Vibration Alarm Capabilities”, which is herein incorporated by reference in its entirety. In some embodiments, to program and deliver a bolus, the user will press the “B” or Up arrow key 108 in the upper right hand corner of the RF programmer 12 keypad 102. Each time the Up arrow key 108 is pushed the amount of the audio bolus will increment in either 0.5 units or 1.0 units (depending on what the user programmed as the incremental step on the “audio” screen of the Set-up 1 menu—alternative embodiments may use other increments). In these examples, units are an increment of insulin. Alternative embodiments, may define units to be any fluid volume, such as micro-liters, ccs, or the like, with the volume being dependent on the type of fluid to be infused. If the user exceeds the desired setting he can wait for an error signal, visual indications, such as flashing, icons, or the like, sound indications such as a “raspberry” type sound, buzzing, tones, sound from a MIDI file, sound from an MP3 file, sound from a WAV file, music or the like, or tactile indications, such as vibration, or the like, and/or any combination of the above, and then press the Up arrow key 108 to begin the process again.

When the desired bolus amount is programmed, the user presses the “activate” or ACT key 110 in the lower left corner of the keypad 24 (or keypad 102 on the RF programmer 12). The external infusion device 10 will then confirm the bolus amount. In alternative embodiments, a visual display or vibration may be used instead of or in addition to audible beeps. To deliver the bolus, the user will then press the ACT key 110 again to start delivery of the bolus. Alternatively, the external infusion device 10 may provide an audible indication by speech

The bolus delivery will commence after the user confirms the bolus amount selection by pressing the ACT key 110 once again. To cancel this bolus before it starts, the user may either allow the external infusion device 10 to time out and return to the time display or press the Down arrow key 112. Either of these will be accompanied by a “raspberry” type beep, and/or other indications as described above, indicating the bolus has been cleared. Preferably, a standard time-out delay of 15 seconds applies to all key presses involved during the bolus amount selection, but other time periods may be used.

In particular embodiments, a BOLUS element, the word DELIVERY, and the updated amount delivered will be displayed on the LCD 28 while delivery is in progress. The external infusion device 10 will beep once, and/or provide other indications as described above, at the end of the dose.

In particular embodiments, an infusion device includes an alarm to notify the user of a potentially missed meal bolus at predetermined times of the day. The user may set the alarm to go once at a particular time. Alternatively, the user may set the alarm to go off every day at a particular time, on alternating days, specific days of the week, specific days of the month or specific days of the year. The alarms may be repetitive, or one time only that requires resetting of the alarm after each use. The alarm may include a snooze function with the duration either factory set or user set. Examples of alarms may be found in U.S. Pat. No. 6,554,798 issued on Apr. 29, 2003 to Mann et al., and entitled “External Infusion Device with Remote Programming, Bolus Estimator and/or Vibration Alarm Capabilities,” U.S. patent application Ser. No. 10/996,136 filed Nov. 22, 2004 and entitled “Improved Infusion Device Menu Structure and Method of Using the Same,” and U.S. patent application Ser. No. 10/025,052 filed Dec. 19, 2001, and entitled “Medication Delivery System and Monitor,” which are herein incorporated by reference in their entirety. The alarm may be provided as a visual indications, such as flashing, icons, or the like, sound indications such as a “raspberry” type sound, buzzing, tones, sound from a MIDI file, sound from an MP3 file, sound from a WAV file, music or the like, or tactile indications, such as vibration, or the like, and/or any combination of the above.

To avoid the nuisance of an unnecessary alarm, the infusion device will check for a predetermined length of time from the time a bolus is being programmed to determine if a missed bolus alarm is scheduled to occur. If an alarm is scheduled to go off during the predetermined length of time, the infusion device will query the user at the time of programming to have the user decide whether to inactivate the alarm at the time the bolus is administered or allow the alarm to notify the user at the scheduled time. This provides a convenient way to deactivate a pending alarm while the user is already engaged and working with the infusion device so that an unnecessary alarm is not sounded at a predetermined time in the future. However, it does not automatically deactivate the alarm, since it requires the user to interact and affirmatively determine to deactivate the alarm. Thus, it avoids the potential of accidentally automatically inactivating an alarm that still should be given (i.e., when a correction bolus is given rather than a meal bolus or vice versa).

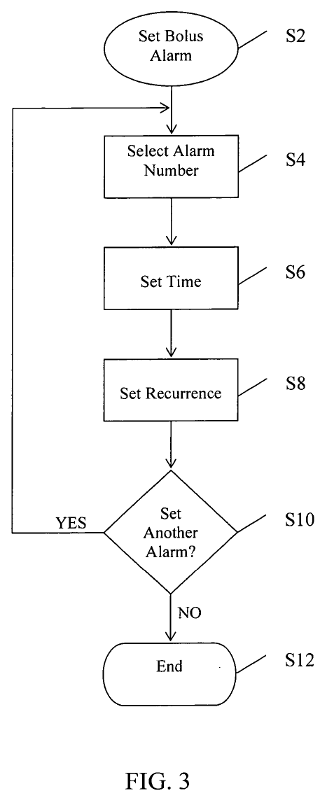

FIG. 3 shows a flow diagram of setting the bolus alarm S2 in accordance with an aspect of an embodiment of the invention. In step S4, the user selects which of one or more alarms to set. In particular embodiments, the user may have only a single alarm to set. In other embodiments, the user has several alarms to select from. In some embodiments there can be 4 or more alarms available. In still further embodiments, the number of alarms can range from 2 to over 100 alarms.

Once the alarm to be set is selected in step S4, the user sets the time for the alarm at step S6. The time is chosen to be the latest point in time at which the user wants to be reminded to check to see if a bolus was taken. In particular embodiments, the time is set in a.m./p.m. increments, and in other embodiments 24 hour time is used.

After the time has been set in step S6, the infusion device checks to see if the user wants to set additional alarms. If yes, the infusion device returns to step S4, and if no the infusion device ends the alarm setting screen at step S12. In alternative embodiments, the user may be asked to set the recurrence of the alarm, as described above. In still other alternative embodiments, the user may be asked to set a specific day and/or date for the alarm. Users may also program an alarm to be delivered as an a alarm in a remote location, either directly or through a relay, or provide an alarm to both the infusion device and the remote location. Examples of alarms being sent to remote locations or using relays can be found in U.S. Pat. No. 6,554,798 issued on Apr. 29, 2003 to Mann et al., and entitled “External Infusion Device with Remote Programming, Bolus Estimator and/or Vibration Alarm Capabilities”, which is herein incorporated by reference in its entirety. In other embodiments, the alarms may be set for different days of the week, different meal times, morning, evening or afternoon, or the like. In still other embodiments, the alarms may be programmed on a different device, such as a remote commander, PDA, personal computer, lap top, cellular telephone or the like, and then downloaded to the infusion device.

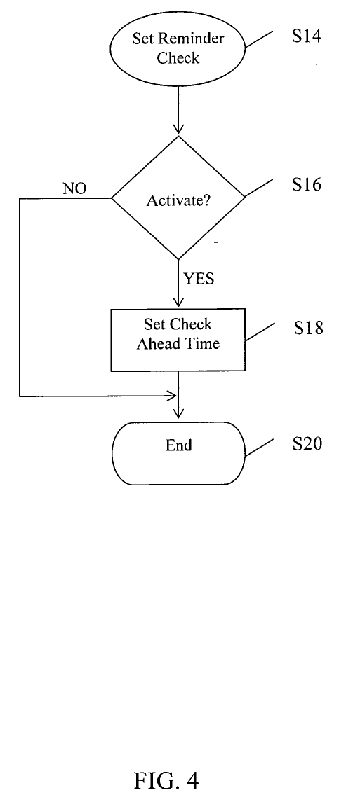

FIG. 4 shows a flow diagram of setting the reminder check S14 in accordance with an aspect of an embodiment of the present invention. In step S16, the user determines whether to activate the bolus reminder check. If the user elects to activate the bolus reminder check in step S16, the user is asked to set a check ahead time in step S118, and the infusion device moves to the end in step S20. If the user selects not to activate the bolus reminder check in step S116, the infusion device moves to end in step S20.

The check ahead time is the time that infusion device looks ahead from the time a bolus is programmed for delivery to see if a bolus reminder alarm has been set. For example, but not limited to, if the check ahead time is set for 2 hours, the infusion device will check to see if a bolus reminder alarm is scheduled to activate at any time up to two hours in the future from the time the user starts to set a bolus for delivery. In particular embodiments, a default value of 2 hours is set. However in alternative embodiments, no default value is set or values ranging from 15 minutes to 6 hours may be used. In other alternatives, the user may be able to customize the specific interval desired. In some embodiments, step S18 is omitted, and a fixed, factory set default value is set. In further embodiments, the bolus reminder check aspect may be set independently for each alarm set by the user so that different and/or the same check ahead time may be used with different alarms. In other embodiments, the check ahead time may be set for different days of the week, different meal times, morning, evening or afternoon, or the like. In still other embodiments, the bolus reminder check may be programmed on a different device, such as a remote commander, PDA, personal computer, lap top, cellular telephone or the like, and then downloaded to the infusion device.

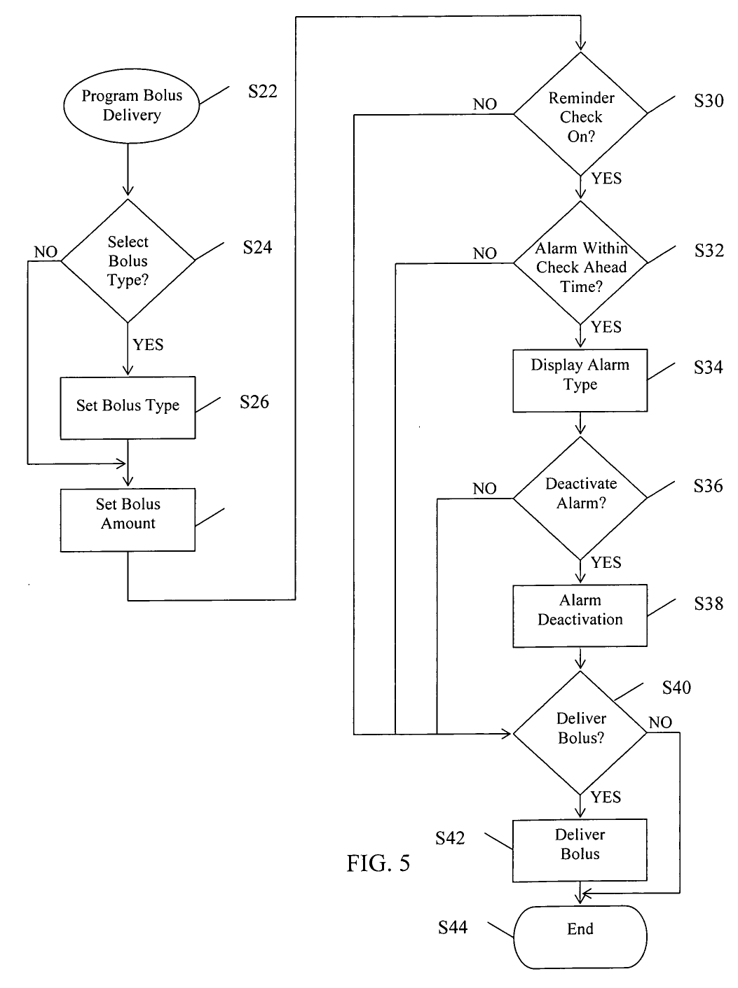

FIG. 5 shows a flow diagram of administering the bolus S22 in accordance with an aspect of an embodiment of the present invention. In step S24, the user determines if they want to select a bolus type (such as normal, square wave, dual wave, custom, audio, profiled, or the like as can be found in U.S. Pat. No. 6,554,798 issued on Apr. 29, 2003 to Mann et al., and entitled “External Infusion Device with Remote Programming, Bolus Estimator and/or Vibration Alarm Capabilities”, which is herein incorporated by reference in its entirety). In some embodiments, step S24 may be omitted. If the user decides to select a bolus type, the user will set the bolus type in step S26, and then moves to step S28 to set the bolus amount for the type of bolus selected. If the user determines not to select a bolus type in step S24, the user sets the bolus amount for the default bolus type (or alternatively the last used type) in step S28. In step S30, the infusion device determines if the reminder check is on.
1. If the reminder check is not on, the infusion device moves to step S40 and asks the user to determine if a bolus should be delivered; or
2. If the reminder check is on, the infusion device moves to step S32 and determines if there are any alarms scheduled to occur during the check ahead time that extends from the current bolus delivery time (and/or time of programming):
   a. If no alarm is scheduled during the check ahead time, the infusion device moves to step S40 and checks with the user to determine if a bolus should be delivered; or
   b. If there is an alarm scheduled during the check ahead time, the infusion device displays the scheduled alarm time in step S34 and then moves to step S36, where it checks with the user to determine if they want to deactivate the scheduled alarm in step S36:
      i. If the user does not want to deactivate the alarm, the infusion device moves to step S40 and asks the user to determine if a bolus should be delivered; or
      ii. If the user determines that the alarm should be deactivated in step S36, the infusion device deactivates the alarm in step S38.

In particular embodiments, the alarm for that time period within the check ahead time is deactivated. In alternative embodiments, the alarm for all future occurrences are deactivated (the user may be queried about this option). In further alternatives, steps S32-S38 may be repeated if multiple alarms are found to occur within the check ahead time from the current bolus time.

In step S40, the user determines if bolus delivery should proceed. If the user decides not to deliver the bolus in step S40, the infusion device ends the delivery at step S42. If the user decides to deliver the bolus in step S40, the infusion device moves to step S42 and delivers the selected bolus. In alternative embodiments, if the bolus is not delivered, the alarm is not deactivated. In other alternatives, if the alarm is deactivated, and the user determines not to deliver a bolus, the alarm is reactivated for the time.

In particular embodiments, all programming is done on the infusion device. In alternative embodiments, the programming may be on the infusion device and/or another device, such as a remote commander, PDA, personal computer, lap top, cellular telephone or the like, and then downloaded to the infusion device.

In further alternative embodiments, the alarm deactivation and method may be applied to other types of alarms. For instance, these embodiments may be used for missed glucose test strip alarms; missed glucose calibration alarms; time to change infusion set alarms; resume infusion device delivery alarms; download infusion device (or other device) data alarms. Accordingly, the embodiments may be applied to alarms that are scheduled to activate in the future, but for which it may be appropriate to deactivate at the time the other activity is being performed.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A medical device for use with a patient, the medical device comprising:
at least one programmable medical device feature;
at least one alarm to remind the patient about the at least one programmable medical device feature; and
at least one reminder check to allow the patient to deactivate the at least one alarm while the at least one programmable medical device feature is being programmed,
wherein the at least one reminder check determines if the at least one alarm is scheduled to occur during a check ahead time that extends from the time the at least one programmable medical device feature is being programmed.

2. The medical device according to claim 1, wherein the at least one programmable medical device feature is at least one fluid delivery.

3. The medical device according to claim 2, wherein the at least one fluid delivery is at least one bolus delivery.

4. The medical device according to claim 3, wherein the at least one bolus delivery includes delivery of at least one of a normal bolus, square wave bolus, dual wave bolus, custom bolus, audio bolus, and/or profiled bolus.

5. The medical device according to claim 3, wherein the at least one alarm is at least one missed bolus alarm.

6. The medical device according to claim 1, wherein the medical device is an infusion device for infusion of fluid to a body of a patient.

7. The medical device according to claim 6, wherein the programmable medical device feature is fluid delivery.

8. The medical device according to claim 7, wherein the fluid is insulin.

9. The medical device according to claim 7, wherein the at least one fluid delivery is at least one bolus delivery.

10. The medical device according to claim 9, wherein the at least one bolus delivery includes delivery of at least one of a normal bolus, square wave bolus, dual wave bolus, custom bolus, audio bolus, and/or profiled bolus.

11. The medical device according to claim 9, wherein the at least one alarm is at least one missed bolus alarm.

12. The medical device according to claim 1, wherein the medical device is an infusion device further including:
a drive mechanism;
a processor to control the infusion device;
a memory operatively coupled to the processor;
at least one power supply;
an input device operatively coupled to the processor to allow the patient to command the processor;
a display device operatively coupled to the processor to provide visual information to the patient; and
a housing.

13. The medical device according to claim 12, wherein the at least one programmable medical device feature is at least one fluid delivery.

14. The medical device according to claim 13, wherein the at least one fluid delivery is at least one bolus delivery.

15. The medical device according to claim 14, wherein the at least one bolus delivery includes delivery of at least one of a normal bolus, square wave bolus, dual wave bolus, custom bolus, audio bolus, and/or profiled bolus.

16. The medical device according to claim 14, wherein the at least one alarm is at least one missed bolus alarm.

17. The medical device according to claim 1, wherein the medical device is a glucose monitor.

18. The medical device according to claim 1, wherein the medical device is a glucose meter.

19. The medical device according to claim 1, wherein the medical device is a PDA used as a programmer for the medical device.

20. A method of deactivating an alarm in a medical device, the method comprising the steps of:
programming at least one programmable medical device feature;
accessing at least one reminder check to determine if at least one alarm is scheduled to occur during a check ahead time that extends from the time the at least one programmable medical device feature is being programmed; and
deactivating the at least one alarm prior to completing programming of the at least one programmable medical device feature.

21. The method according to claim 20, wherein the at least one programmable medical device feature is at least one fluid delivery.

22. The method according to claim 21, wherein the at least one fluid delivery is at least one bolus delivery.

23. The method according to claim 22, wherein the at least one bolus delivery includes delivery of at least one of a normal bolus, square wave bolus, dual wave bolus, custom bolus, audio bolus, and/or profiled bolus.

24. The method according to claim 22, wherein the at least one alarm is at least one missed bolus alarm.

25. The method according to claim 20, wherein the method is for deactivating a missed bolus alarm in an infusion device.

26. A medical device for use with a patient, the medical device comprising:
programming means for controlling at least one programmable medical device feature in the medical device;
alarm means for reminding the patient about the at least one programmable medical device feature; and
reminder means for allowing the patient to deactivate the alarm means while the programming means is being used to program the at least one programmable medical device feature,
wherein the reminder means determines if the alarm means is scheduled to occur during a check ahead time that extends from the time the programming means is programming the at least one medical device feature.

27. The medical device according to claim 26, wherein the medical device is an infusion device,
wherein the programming means includes bolus means for programming at least one bolus delivery as the at least one programmable medical device feature,
wherein the alarm means includes missed bolus alarm means for notifying the patient of a missed bolus delivery,
wherein the reminder means is for allowing the patient to deactivate the missed bolus alarm means while the at least one bolus delivery is being programmed, and
wherein the reminder means determines if the missed bolus alarm means is scheduled to occur during a check ahead time that extends from the time the at least one bolus delivery is being programmed.
